(19) United States
(12) Patent Application Publication
(10) Pub. No.: US 2017/0021346 A1
(43) Pub. Date: Jan. 26, 2017

(54) FREEZE-DRYING OF ORGANOALUMINUM CO-CATALYST COMPOSITIONS AND TRANSITION METAL COMPLEX CATALYST COMPOSITIONS

(71) Applicant: SAUDI BASIC INDUSTRIES CORPORATION, Riyadh (SA)

(72) Inventor: Roland SCHMIDT, Wiehl (DE)

(21) Appl. No.: 15/300,884

(22) PCT Filed: Apr. 6, 2015
(86) PCT No.: PCT/IB2015/052481

§ 371 Date: Sep. 30, 2016

Related U.S. Application Data
(60) Provisional application No. 61/976,120, filed on Apr. 7, 2014.

(51) Int. Cl. B01J 37/32; B01J 31/22; B01J 31/14; C07C 2/32; B01J 31/02

(52) U.S. Cl. CPC B01J 37/32; C07C 2/32; B01J 31/0204; B01J 31/143; B01J 31/2226; C07C 2531/14; C07C 2531/16; B01J 2231/20; B01J 2531/46

(57) ABSTRACT

Processes of preparing freeze-dried co-catalyst compositions are provided. In an exemplary embodiment, the process includes mixing an organoaluminum compound with a modifier at low temperature to provide a modified co-catalyst composition. The process further includes further cooling the modified co-catalyst composition under reduced pressure, to provide a freeze-dried co-catalyst composition. Processes of preparing freeze-dried catalyst compositions, processes of preparing catalyst compositions, freeze-dried co-catalyst compositions, freeze-dried catalyst compositions, catalyst compositions, and processes of preparing α-olefins are also provided.

### FIELD

The presently disclosed subject matter relates to methods of preparing freeze-dried organoaluminum co-catalyst compositions and transition metal catalyst compositions that can be used in olefin oligomerization processes and other processes.

### BACKGROUND

Various catalytic processes involve co-catalysts based on organoaluminum compounds such as TEAL (triethylaluminum), EASC (ethylaluminum sesquichloride), and others. Such co-catalysts may be used in conjunction with other catalysts, e.g., transition metal complexes, to perform various catalytic processes and promote various chemical reactions. Organoaluminum co-catalysts are used for olefin (alkene) oligomerization processes. For example, organoaluminum co-catalysts can be used in conjunction with transition metal complexes to produce catalyst compositions capable of oligomerizing ethylene (ethene) to 1-butene. Organoaluminum co-catalysts are also used in various olefin polymerization processes. For example, organoaluminum co-catalysts can be used in conjunction with transition metal complexes to produce catalyst compositions capable of generating polyethylene, polypropylene, and other polymers. Organoaluminum compounds can also be used, alone or in conjunction with transition metal complexes, to achieve many other chemical transformations, e.g., diene and alkyne polymerization processes, generation of alkylidene and metal-carbene complexes, transmetallation reactions, carbometallation reactions of alkenes, dienes, and alkynes, and conjugate addition reactions.

1-Butene is an example of a material that can be generated using catalytic processes involving co-catalysts based on organoaluminum compounds. 1-Butene, also known as 1-butylene, α-butene, and α-butylene, has for a long time been a desirable substance in the chemical industry. Not only can 1-butene be converted to polybutene-1 and butylene oxides, it can also be used as a co-monomer with ethylene for the production of high strength and high stress crack resistant polyethylene resins. The major industrial routes for producing 1-butene include steam cracking of C₄ hydrocarbon streams, ethylene oligomerization processes, refinery operations of crude oil and ethylene dimerization processes. Catalytic dimerization of ethylene into 1-butene produces higher chain polymers via the growth reaction of the organoaluminum compounds (Ziegler, Angew. Chem. (1952); 64:323-329; J. Boor, Editor, Ziegler-Natta Catalysts and Polymerizations, Acad. Press (New York) 1979; Handbook of Transition Metal Polymerization Catalysts, R. Hoff, R. T. Mathers, Eds. 2010 John Wiley & Sons).

One route to the preparation of 1-butene is the cracking of higher petrochemical fractions containing more than four carbon atoms. A further route to the preparation of 1-butene is via the catalytic dimerization of ethylene. The industrial synthesis of 1-butene can be achieved using nickel or titanium catalysts in large industrial processes such as Alphabutol™ (Handbook of Petroleum Processing, Edited by D. S. J. Jones, P. R. Pujadó; Springer Science 2008; Forestière et al., Oil & Gas Science and Technology-Rev. IFP (2009); 64(6):649-667).

In the Alphabutol™ system and other existing processes of preparation of 1-butene by catalytic dimerization of ethylene, catalyst compositions are formed by combining organoaluminum co-catalysts with transition metal complexes. For example, a solution of an organoaluminum co-catalyst in a hydrocarbon solvent can be mixed with a solution of a titanium complex in an ether solvent to obtain a catalyst composition, which is used to prepare 1-butene, as in the Alphabutol™ system. Such catalyst systems can suffer from drawbacks, which include low catalyst activity, a lengthy induction period, and process fouling, including precipitation of polyethylene. The catalytic activity of the Alphabutol™ system can be relatively low at roughly 1 kg of product per gram of titanium. Polymer formation and lengthy initial induction period are major drawbacks for the commercial Alphabutol™ system. Also, the Alphabutol™ system and other existing processes of preparation of 1-butene can involve catalyst compositions with poorly defined stoichiometry (e.g., ratios of organoaluminum co-catalyst to transition metal complex to ether), which can affect reproducibility and other reaction characteristics.

Polyethylene is another example of a material that can be generated using catalytic processes involving co-catalysts based on organoaluminum compounds. Polyethylene is a highly valuable plastic used in a wide variety of applications. Polyethylene is commonly generated via Ziegler-Natta polymerization of ethylene. Existing Ziegler-Natta catalyst systems can suffer from drawbacks, including a need for very large excesses of organoaluminum co-catalyst. Also, existing Ziegler-Natta catalyst systems can involve catalyst compositions with poorly defined stoichiometry (e.g., ratios of organoaluminum co-catalyst to transition metal complex), which can affect reproducibility and other reaction characteristics.

Both homogeneous catalysts and heterogeneous catalysts are known to effect various oligomerization reactions of alkenes, e.g., dimerization of ethylene to form 1-butene. Both homogeneous catalysts and heterogeneous catalysts are also known to effect various polymerization reactions of alkenes, e.g., Ziegler-Natta polymerization of ethylene to form polyethylene. Heterogeneous catalysts include catalyst compositions that include transition metal complexes affixed to a solid support or solid carrier. While both homogeneous and heterogeneous catalysts can have benefits and drawbacks, homogeneous catalysts can have advantages over heterogeneous catalysts in certain contexts. For example, homogeneous catalysts can be more readily characterized and “fine-tuned” to achieve optimal reaction conditions. Also, heterogeneous catalysts that include transition metal complexes affixed to a solid support or solid carrier can suffer declines in catalytic activity over time, as active centers on the heterogeneous catalysts can become plugged or blocked by polymers, various side products, and other materials.

Various methods are known for purification of catalysts, e.g., crystallization, washing, evaporation, and freeze-drying. Freeze-drying is a method of separating relatively volatile substances from less volatile substances. Freeze-drying is also known as lyophilization and cryodessication. Freeze-drying involves freezing a sample of material, which can involve cooling to low temperature, and exposing the frozen material to reduced pressure (vacuum) so that volatile substances vaporize in the vacuum (sublime) without melting while less volatile substances remain solid in the frozen material. Freeze-drying is commonly performed to remove water from a material, but freeze-drying can also be used to remove other volatile substances (e.g., non-aqueous solvents and impurities) from a material.

There remains a need in the art for co-catalyst compositions and catalyst compositions that are suitable for various processes, including various homogeneous catalytic reactions, e.g. dimerization of ethylene, and that are characterized by improved catalytic activity, shortened induction period, long lifetimes, and high selectivity.

### SUMMARY

The presently disclosed subject matter provides processes of preparing freeze-dried co-catalyst compositions. In some embodiments, a process includes mixing an organoaluminum compound with a modifier at low temperature, to provide a modified co-catalyst composition. The process further includes further cooling the modified co-catalyst composition under reduced pressure, to provide a freeze-dried co-catalyst composition.

The presently disclosed subject matter also provides processes of preparing freeze-dried catalyst compositions. In some embodiments, a non-limiting example process includes mixing an organoaluminum compound with a transition metal complex and a modifier at low temperature, to provide a modified catalyst composition. The process further includes cooling the modified catalyst composition under reduced pressure, to provide a freeze-dried catalyst composition.

The presently disclosed subject matter also provides processes of preparing catalyst compositions. In some embodiments, a non-limiting example process includes mixing an organoaluminum compound with a modifier at low temperature, to provide a modified co-catalyst composition. The process further includes further cooling the modified co-catalyst composition under reduced pressure, to provide a freeze-dried co-catalyst, and mixing the freeze-dried co-catalyst composition with a transition metal complex, to provide a catalyst composition.

The presently disclosed subject matter also provides compositions prepared by the above-described processes. In some embodiments, a freeze-dried co-catalyst composition is prepared by the above-described process of preparing a freeze-dried co-catalyst composition. In another embodiment, a freeze-dried catalyst composition is prepared by the above-described process of preparing a freeze-dried catalyst composition. In another embodiment, a catalyst composition is prepared by the above-described process of preparing a catalyst composition.

The presently disclosed subject matter also provides freeze-dried co-catalyst compositions. In some embodiments, a non-limiting example composition includes an organoaluminum compound and a modifier. In certain embodiments, the modifier can be a compound that decreases the initial reducing strength of the organoaluminum compound. The modifier can be an ether. The ether can be tetrahydrofuran (THF). In certain embodiments, the composition does not include a solid support or solid carrier.

The presently disclosed subject matter also provides freeze-dried catalyst compositions. In some embodiments, a non-limiting example composition includes an organoaluminum compound, a transition metal complex, and a modifier. In certain embodiments, the transition metal complex is titanium tetra-n-butoxide. In certain embodiments, the composition does not include a solid support or solid carrier.

The presently disclosed subject matter also provides processes of preparing an α-olefin. In some embodiments, a non-limiting example process includes providing a freeze-dried catalyst composition including an organoaluminum compound, a transition metal complex, and a modifier. The process further includes contacting an alkene with the freeze-dried catalyst composition to obtain an α-olefin.

In certain embodiments, the process of preparing an α-olefin can further include dissolving the freeze-dried catalyst composition in a solvent prior to contacting the alkene with the freeze-dried catalyst composition.

The presently disclosed subject matter also provides processes of preparing a polymer. In some embodiments, a non-limiting example process includes providing a freeze-dried catalyst composition including an organoaluminum compound, a transition metal complex, and a modifier. The process further includes contacting an alkene with the freeze-dried catalyst composition to obtain a polymer.

In certain embodiments, the process of preparing a polymer can further include dissolving the freeze-dried catalyst in a solvent prior to contacting the alkene with the freeze-dried catalyst composition.

### BRIEF DESCRIPTION OF THE DRAWINGS

FIG. 1 is a flow diagram illustrating a process of preparing a freeze-dried co-catalyst composition, in accordance with one non-limiting exemplary embodiment.

FIG. 2 is a flow diagram illustrating a process of preparing a freeze-dried catalyst composition, in accordance with one non-limiting exemplary embodiment.

FIG. 3 represents the dimeric and monomeric forms of triethylaluminum.

### DETAILED DESCRIPTION

The presently disclosed subject matter provides processes of preparing organoaluminum co-catalyst compositions and transition metal complex catalyst compositions. The processes involve freeze-drying. The disclosed catalyst and co-catalyst compositions can be used in olefin oligomerization processes, e.g., catalytic dimerization of ethylene (ethene) to produce 1-butene, and other processes.

Freeze-drying can have advantages over other methods of separating relatively volatile substances from less volatile substances. For example, freeze-drying can be conducted at low temperature, which can preserve the integrity of materials that can decompose or otherwise degrade at elevated temperatures. By contrast, evaporation of volatile liquids at ambient pressure often requires application of heat, which can cause degradation. Freeze-drying can be conducted in a vacuum, in conditions with very low levels of moisture (water) and oxygen, which can preserve the integrity of materials that are sensitive to moisture and oxygen. The solid products of freeze-drying can be of higher purity than the crude material initially subjected to the freeze-drying process, and the products of freeze-drying can, in certain contexts, be stored under appropriate conditions for days, weeks, months, or years.

The catalyst compositions disclosed herein include a transition metal complex and an organoaluminum compound. In the catalyst composition, the transition metal complex can be the main catalyst, and the organoaluminum compound can be a co-catalyst or an activator to activate the transition metal complex. The organoaluminum compound can activate the transition metal complex via reduction reactions, i.e., the organoaluminum compound can be a reducing agent for the transition metal complex. For example, the organoaluminum compound can transfer electrons to the metal center of the transition metal complex. The transition metal complex can be reduced by the organoaluminum compound to various oxidation states. Some of the reduced transition metal complexes are beneficial and some are not, depending on the nature of the chemical reaction being catalyzed. The organoaluminum compound may also act as a co-catalyst or an activator of the transition metal complex by releasing free coordination sites on the metal center of the transition metal complex and/or by exchanging ligands with the transition metal complex to generate organotransition metal bonds.

In the catalyst compositions, the organoaluminum compound and the transition metal complex can be mixed in various molar ratios. For example, the molar ratio of organoaluminum compound to transition metal complex can be less than about 1:1, in a range from about 1:1 to about 3:1, in a range from about 3:1 to about 6:1, or greater than about 6:1. In certain non-limiting embodiments, the molar ratio of organoaluminum compound to transition metal complex can be in a range from 1:1 to 3:1, e.g., about 2:1. The molar ratio of organoaluminum compound to transition metal complex can have an effect on the catalytic activity of the catalyst composition. For example, in certain non-limiting embodiments, when the transition metal complex is a complex of titanium, catalyst compositions having an organoaluminum compound to titanium complex molar ratio of less than about 10:1 can show selectivity for oligomerization of ethylene, while catalyst compositions having an organoaluminum compound to titanium complex molar ratio of greater than about 20:1 can show selectivity for polymerization of ethylene.

As noted above, a transition metal complex can be the main catalyst in a catalyst composition that further includes an organoaluminum compound as co-catalyst. The transition metal complex disclosed herein can include at least one of the metals of Groups IV-B, V-B, VI-B, VII-B, and VIII of the Periodic Table. The suitable metals include, but are not limited to, titanium, zirconium, hafnium, vanadium, niobium, tantalum, chromium, molybdenum, tungsten, iron, cobalt, nickel, palladium, platinum, and a combination thereof. The transition metal complex can be an alkyl titanate having a general formula of Ti(OR)₄, where R is a linear or branched alkyl radical having from about 1 to about 12 carbon atoms, e.g., a C₂-C₁₂ alkyl group, a C₂-C₈ alkyl group, or a C₃-C₅ alkyl group, or respective aromatic moieties. In some embodiments, the alkyl group is butyl. In certain embodiments, the transition metal complexes include titanium. Suitable transition metal complexes including titanium include, but are not limited to, tetraethyl titanate, tetraisopropyl titanate, titanium tetra-n-butoxide (TNBT), and tetra-2-ethyl-hexyl titanate. In some embodiments, the transition metal complex is titanium tetra-n-butoxide.

The transition metal complex can be present in high concentration in the catalyst composition. In some embodiments, the transition metal complex is present in a concentration of from about 0.0001 to about 0.1 mol/dm³, from about 0.0001 to about 0.0005 mol/dm³, from about 0.0005 to about 0.001 mol/dm³, from about 0.001 to about 0.01 mol/dm³, from about 0.01 to about 0.1 mol/dm³.

In certain embodiments, the transition metal complex can be diluted or dissolved in a solvent, which includes, but is not limited to, hydrocarbon solvents including alkanes (including C₂-C₁₂ alkanes or C₄-C₈ alkanes, e.g., pentane, hexane, heptane, octane), aromatic hydrocarbons (benzene, toluene), and olefins or alkenes (including C₂-C₁₂ alkenes or C₄-C₈, e.g., 1-butene, pentenes, hexenes). In some embodiments, the transition metal complex is diluted in hexane. In other embodiments, the transition metal complex can be first mixed or reacted with a modifier, which can be an ether (e.g., diethyl ether, THF, or 1,4-dioxane) or another polar additive capable of coordinating to the transition metal.

As noted above, an organoaluminum compound can be a co-catalyst or an activator to activate a transition metal complex in a catalyst composition. The organoaluminum compound can have the general formula of Al(R)₃, where R can be a hydrocarbon, H or a halogen, or mixtures thereof. Each R in a molecule may be the same as or different to the other R groups in the molecule. Organoaluminum compounds are known to one of ordinary skill in the art, and the artisan can select the organoaluminum compounds in order to enhance the advantageous properties of the process according to the presently disclosed subject matter. In some embodiments, R is an alkyl group. R can be a straight chain or branched alkyl group. In some embodiments, R is a straight chain alkyl group. R can be a C₁-C₁₂ alkyl group, a C₁-C₈ alkyl group, a C₁-C₄ alkyl group. In some embodiments, the alkyl group is ethyl. Suitable organoaluminum compounds include, but are not limited to, triethylaluminum (TEAL), trimethylaluminum (TMA), tripropylaluminum, triisobutylaluminum, diisobutylaluminum hydride, ethylaluminum sesquichloride (EASC), and trihexylaluminum. In some embodiments, the organoaluminum compound is aluminum trialkyls, which can be triethylaluminum and trimethylaluminum. Aluminum trialkyls exist in both dimeric form and monomeric form.

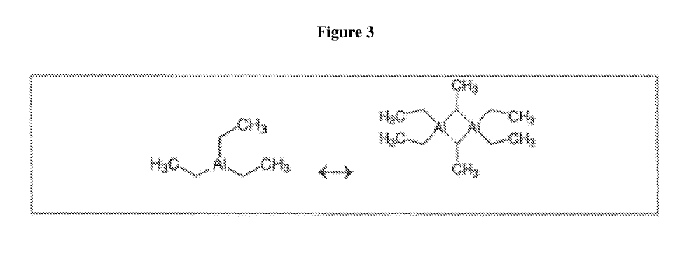

In some embodiments, the organoaluminum compound is TEAL. TEAL is a volatile, colorless and highly pyrophoric liquid. TEAL can be stored in hydrocarbon solvents such as hexane, heptane, or toluene. TEAL exists in dimeric form as Al₂Et₆ and monomeric form as AlEt₃, where Et is an ethyl (CH₂CH₃) group (see FIG. 3). One pair of ethyl groups is bridging and four ethyl groups are terminal ligands as shown in FIG. 3. At higher temperatures, the dimer Al₂Et₆ cracks into the monomer AlEt₃.

In another embodiment, the organoaluminum compound is TMA. Similar to TEAL, TMA is a pyrophoric and colorless liquid. TMA also exists in dimeric form as Al₂Me₆ and monomeric form as AlMe₃, where Me is a methyl (CH₃) group. TMA exists mostly as a dimer at ambient temperature and pressure. The shared methyl groups bridge between the two aluminum atoms (3-centered-2-electron bonds) tend to undergo reactions with Lewis bases that would give products consisting of 2-centered-2-electron bonds. For instance, R₃N—AlMe₃ can be obtained upon treating the TMA dimer with amines. (AlMe₂Cl)₂ can be obtained upon treating a TMA dimer with aluminum trichloride. TMA monomer AlMe₃, which has an aluminum atom bonded to three methyl groups, usually exists at high temperature and low pressure.

The processes disclosed herein can include modifying with a modifier the organoaluminum compound useful as a co-catalyst. The modifier can be a chemical species that modifies the initial reducing strength of the organoaluminum compound, i.e., a reduction modifier. The reducing strength of a given organoaluminum compound can be an important factor in the behavior of a catalyst system including an organoaluminum compound and a transition metal complex, as the organoaluminum compound can activate the transition metal complex by reduction to form catalytically active species. For example, an organoaluminum compound can activate an alkyl titanate to generate a catalyst composition to produce 1-butene from catalytic dimerization of ethylene. However, due to the strong reducing strength of the organoaluminum compound, it can deactivate the activated transition metal complex via further reduction reactions. The activated transition metal complex can be deactivated to various inactive species, including, but not limited to, various mixed oxidation state complexes of the transition metal and aluminum. Without being bound to any particular theory, it can be that, for example, titanium complexes can be deactivated when reduced to low oxidation states including Ti(I) and Ti(II), which are relatively ineffective as catalysts.

In the presence of the modifier, the organoaluminum compound primarily exists in monomeric form. The organoaluminum compound monomer can coordinate to the modifier. In the presence of the modifier, e.g., THF, the polarity of the solvent around the organoaluminum compound increases. The modifier can lower or decrease the initial reducing strength of the organoaluminum compound (“taming” it), thereby discouraging over-reduction of the transition metal complex to inactivated species and/or discouraging the deactivation of the activated transition metal complex.

The catalyst compositions disclosed herein can be used for catalytic dimerization of ethylene, e.g., to produce 1-butene. Modifying the organoaluminum compound with the modifier prior to mixing the organoaluminum compound with the transition metal complex can improve the overall catalytic activity of the transition metal complex. In the commercial Alphabutol™ system, the main catalyst is TNBT and the co-catalyst is TEAL. The catalytic activity can be measured or evaluated based on the total ethylene consumption in the catalytic dimerization of ethylene by using a catalyst composition, e.g., the presently disclosed catalyst composition or the commercial Alphabutol™ system.

In the commercial Alphabutol™ system, the main catalyst TNBT is first mixed or reacted with a catalyst modifier. TEAL can then be added to activate TNBT. Catalyst modifiers can be polar additives and can coordinate to TNBT with a pair of electrons, thereby effecting changes in the nature of active metal centers and having a profound effect on the catalyst activity and selectivity. The catalyst modifier can be an ether, e.g., THF. In some embodiments of the presently disclosed catalyst composition, the transition metal complex is not mixed with and is free of a catalyst modifier (e.g., THF). In this example, the transition metal complex is instead diluted in a solvent, which includes, but is not limited to, alkanes (including C₂-C₁₂ alkanes or C₄-C₈ alkanes, e.g., pentane, hexane, heptane, octane), aromatic hydrocarbons (benzene, toluene), and olefins or alkenes (including C₂-C₁₂ alkenes or C₄-C₈, e.g., 1-butene, pentenes, hexenes). In some embodiments, the transition metal complex is diluted in hexane.

The modifier can be various compounds capable of modifying an organoaluminum compound. For example, the modifier can be an aprotic compound possessing lone pair electrons capable of coordinating to an organoaluminum compound. The modifier can be a compound that decreases the initial reducing strength of an organoaluminum compound. The reaction modifier can be an ether, an anhydride, an amine, an amide, a silicate, a silyl ether, a siloxane, an ester, a carbonate, a carbamate, a sulfoxide, a sulfone, a phosphoramide, a silane, an acetal, or a combination thereof.

In some embodiments, the modifier is an ether. The ether can be a monoether or poly-ethers including at least two ether groups. Substituents of the ether can be alkyl, aryl, or other groups. Suitable alkyl groups can be methyl, ethyl, propyl, n-butyl, iso-butyl, t-butyl, and other higher alkyl groups. In some embodiments, the ether is a monoether. Suitable monoethers include, but are not limited to, diethyl ether, dipropyl ether, dibutyl ether, methyl ethyl ether, methyl propyl ether, methyl butyl ether, methyl tert-butyl ether, ethyl propyl ether, ethyl butyl ether, propyl butyl ether, tetrahydrofuran (THF), and dihydropyran. In some embodiments, the monoether is THF.

In another embodiment, the ether is a polyether that includes at least two ether groups. Suitable polyethers include, but are not limited to, dioxane and ethers based on poly-alcohols, e.g., glycols or glycerols, e.g., ethylene glycol. In some embodiments, the ether is dioxane, which can be 1,4-dioxane. Ethers based on glycol include, but are not limited to, 1,2-dimethyl ethylene glycol ether (1,2-DME or DME), diethyl ethylene glycol ether, dipropyl ethylene glycol ether, dibutyl ethylene glycol ether, methyl ethyl ethylene glycol ether, methyl propyl ethylene glycol ether, methyl butyl ethylene glycol ether, ethyl propyl ethylene glycol ether, ethyl butyl ethylene glycol ether, and propyl butyl ethylene glycol ether.

In some embodiments, the modifier is an anhydride. The anhydride can be acetic acid anhydride.

In some embodiments, the modifier is an amine. Suitable amines include, but are not limited to, diethylamine and triethylamine.

In some embodiments, the modifier is an amide. The amide can be N,N-dimethylacetamide.

In some embodiments, the modifier is a silicate. The silicate can be tetraethylorthosilicate.

In some embodiments, the modifier is a silyl ether. The silyl ether can be (trimethylsilyl)tert-butyl alcohol.

In some embodiments, the modifier is a siloxane. Suitable siloxanes include, but are not limited to, hexamethyldisiloxane and 1,3-bis-tert-butyl-1,1,3,3-tetramethyldisiloxane.

In some embodiments, the modifier is an ester. Suitable esters include, but are not limited to, tert-butyl pivalate and tert-butyl acetate.

In some embodiments, the modifier is a carbonate. The carbonate can be di-tert-butyl carbonate.

In some embodiments, the modifier is a carbamate. The carbamate can be 1,3-dimethyl-3,4,5,6-tetrahydro-2(1H)-pyrimidinone (DMPU).

In some embodiments, the modifier is a sulfoxide. The sulfoxide can be dimethylsulfoxide (DMSO).

In some embodiments, the modifier is a sulfone. The sulfone can be sulfolane.

In some embodiments, the modifier is a phosphoramide. The phosphoramide can be hexamethylphosphoramide.

In some embodiments, the modifier is a silane. The silane can be trimethoxysilane.

In some embodiments, the modifier is an acetal. Suitable acetals include, but are not limited to, dimethoxymethane (DMM, formal, or methylal), diethoxymethane (DEM or ethylal), dibutoxymethane (DBM or butylal), and 1,3-dioxolane (dioxolane),

As noted above, freeze-drying is a method of separating relatively volatile substances from less volatile substances. Freeze-drying involves freezing a sample of material, which can involve cooling to low temperature, and exposing the frozen material to reduced pressure (vacuum) so that volatile substances vaporize in the vacuum (sublime) without melting while less volatile substances remain solid in the frozen material. Freeze-drying can be performed with standard equipment known in the art, e.g., in a freeze-drying apparatus. Examples of freeze-drying apparatuses include manifold freeze-dryers, rotary freeze-dryers, and tray-style freeze-dryers. Freeze-drying can also be conducted on a vacuum gas manifold or Schlenk line.

As noted above, freeze-drying can have advantages over other methods of separating relatively volatile substances from less volatile substances. For example, freeze-drying can be conducted at low temperature, which can preserve the integrity of materials that can decompose or otherwise degrade at elevated temperatures. Organoaluminum compounds and transition metal complexes can degrade at elevated temperature, so avoidance of elevated temperatures can improve catalyst integrity, activity, and lifetime. Freeze-drying can be conducted in a vacuum, in conditions with very low levels of moisture (water) and oxygen, which can preserve the integrity of materials that are sensitive to moisture and oxygen. Organoaluminum compounds and transition metal complexes can be sensitive to moisture and oxygen, so minimization of moisture and oxygen can improve catalyst integrity, activity, and lifetime. The solid products of freeze-drying can be of higher purity than the crude material initially subjected to the freeze-drying process, and the products of freeze-drying can, in certain contexts, be stored under appropriate conditions for days, weeks, months, years, or even longer. The freeze-dried compositions disclosed herein can be stored under inert conditions for days, weeks, months, years, or even longer.

In accordance with some embodiments, a process of preparing a freeze-dried co-catalyst composition includes mixing an organoaluminum compound with a modifier at low temperature to provide a modified co-catalyst composition. The molar ratio of the organoaluminum compound to the modifier can vary in a wide range, e.g., from about 0.1 to about 50. For example, the molar ratio of the organoaluminum compound to the modifier can be from about 0.1 to about 1, from about 1 to about 5, from about 1 to about 10, from about 5 to about 10, from about 10 to about 20, from about 20 to about 30, from about 30 to about 40, or from about 40 to about 50. In some embodiments, the molar ratio of the organoaluminum compound to the modifier is about 1:5. The organoaluminum compound can coordinate to the modifier. Coordination of the organoaluminum compound and the modifier can be marked by evolution of heat, i.e., an exotherm. The mixing of the organoaluminum compound with the modifier can be performed at a temperature of about −50° C. to about 20° C. and a pressure of from about 1 atm to about 250 atm. In certain embodiments, the mixing can be performed at a temperature of about −30° C. to about 10° C. The organoaluminum compound can be diluted in or mixed with the modifier for from about 1 minute to about 1 day. The process can further include bringing the mixture of the organoaluminum compound and the modifier to ambient conditions, e.g., a temperature of about 25° C. and a pressure of about 1 atm. At this point, a modified co-catalyst composition can be obtained.

In certain embodiments, the organoaluminum compound can be diluted in a solvent, which includes, but is not limited to, alkanes (including C₂-C₁₂ alkanes or C₄-C₈ alkanes, e.g., pentane, hexane, heptane, octane), aromatic hydrocarbons (benzene, toluene), and olefins or alkenes (including C₂-C₁₂ alkenes or C₄-C₈, e.g., 1-butene, pentenes, hexenes). The organoaluminum compound can be diluted in a solvent before and/or after it is diluted in or mixed with the modifier. Alternatively, the organoaluminum compound can be used without diluting in a solvent before or after it is diluted in or mixed with modifier.

The exemplary process disclosed herein further includes further cooling the modified co-catalyst composition under reduced pressure, to provide a freeze-dried co-catalyst composition. The further cooling under reduced pressure can constitute a freeze-drying step. The further cooling can be performed at a temperature of about −200° C. to about 0° C. In certain embodiments, the further cooling can be performed at a temperature of about −80° C. to about −50° C. In other embodiments, the further cooling can be performed at a temperature of about −200° C. to about −80° C. In certain embodiments, the further cooling can be performed at liquid nitrogen temperatures, e.g., at about −196° C. The further cooling can be performed at a reduced pressure of less than ambient pressure, i.e., less than 1 atm. In certain embodiments, the pressure can be less than 0.1 atm, less than 0.01 atm, or less than 0.001 atm. During the further cooling under reduced pressure, relatively volatile substances, including excess solvent (e.g., hexane) and modifier (e.g., THF), can be removed via vaporization while the desired co-catalyst composition remains solid.

For the purpose of illustration and not limitation, FIG. 1 shows an exemplary process 101 of preparing a freeze-dried co-catalyst composition. As shown in FIG. 1, the process 101 includes providing an organoaluminum compound 102 and a modifier 103. The organoaluminum compound 102 and the modifier 103 can be mixed at low temperature 104 to provide a modified co-catalyst composition 105. The modified co-catalyst composition 105 can be further cooled under reduced pressure 106 to provide a freeze-dried co-catalyst composition 107.

The freeze-dried co-catalyst compositions, which include an organoaluminum compound and a modifier, can have properties that differ from the properties of unmodified organoaluminum compounds, e.g., lower initial reduction strength. The freeze-dried co-catalyst compositions can be used as a co-catalyst for various catalytic processes. The freeze-dried co-catalyst compositions can generally be used as a co-catalyst under the same reaction conditions as existing organoaluminum compounds. For example, a transition metal complex can be added to the freeze-dried co-catalyst composition in a standard ratio of organoaluminum to transition metal complex to produce a catalytic mixture.

The freeze-dried co-catalyst compositions disclosed herein can have improved properties as compared to other co-catalyst compositions. For example, the freeze-dried co-catalyst compositions can have well-defined stoichiometry (i.e., molar ratio of organoaluminum compound to modifier), as any excess modifier can be removed during freeze-drying. Co-catalyst compositions with well-defined stoichiometry can have improved properties, e.g., improved reproducibility and catalytic activity. Also, the freeze-dried co-catalyst compositions can be purified as compared to non-freeze-dried co-catalyst compositions, as solvent and other impurities can be removed during freeze-drying. The freeze-dried co-catalyst compositions can be waxy or solid in form, which can make them convenient to store and transport. The freeze-dried co-catalyst compositions can be stored at low temperature and under an inert atmosphere. The freeze-dried co-catalyst compositions can be stable to storage for extended periods, e.g., for a week, a month, a year, or longer. After storage, the freeze-dried co-catalyst compositions can be mixed with a transition metal complex under standard conditions to prepare a catalyst composition.

Heterogeneous catalysts can involve solid supports or solid carriers that feature high surface area. Examples of solid supports and solid carriers used to prepare heterogeneous catalysts can include various metal salts, metalloid oxides, and metal oxides, e.g., titanium oxide, zirconium oxide, silica (silicon oxide), alumina (aluminum oxide), magnesium oxide, and magnesium chloride. In certain embodiments, the co-catalyst compositions disclosed herein can be used to prepare homogeneous catalysts rather than heterogeneous catalysts. Accordingly, the organoaluminum compound and the modifier can be mixed and freeze-dried in the absence of any solid support or solid carrier, to provide a composition that does not include a solid support or solid carrier.

In accordance with some embodiments, a process of preparing a freeze-dried catalyst composition includes mixing an organoaluminum compound with a transition metal complex and a modifier at low temperature to provide a modified catalyst composition. The molar ratio of the organoaluminum compound to the modifier can vary in a wide range, as noted above. The molar ratio of the organoaluminum compound to the transition metal complex can also vary in a wide range, as noted above. In certain embodiments, the organoaluminum compound can be mixed first with the modifier, and the transition metal complex can be added subsequently. The mixing of the organoaluminum compound with the modifier and the transition metal complex can be performed at a temperature of about −50° C. to about 20° C. and a pressure of from about 1 atm to about 250 atm. In certain embodiments, the mixing can be performed at a temperature of about −30° C. to about 10° C. The organoaluminum compound can be diluted in or mixed with the modifier for about 1 minute to about 1 day, and the transition metal complex can be diluted in or mixed with the organoaluminum compound and modifier for about 1 minute to about 1 day. In some embodiments, the transition metal complex is not diluted in or mixed with the organoaluminum compound and modifier until shortly before the modified catalyst composition is subjected to freeze-drying. For example, the organoaluminum compound modified by a modifier can be brought into contact with the transition metal complex to activate the latter not earlier than 30 minutes, not earlier than 15 minutes, not earlier than 5 minutes, or not earlier than 3 minutes before freeze-drying. In certain embodiments, the catalyst composition can be kept at low temperature before cooling further during freeze-drying. In other embodiments, the process can include bringing the catalyst composition to ambient conditions, e.g., a temperature of about 25° C. and a pressure of about 1 atm. At this point, a modified catalyst composition can be obtained.

In certain embodiments of the presently disclosed subject matter, when the modified organoaluminum compound and the transition metal complex are combined to produce a catalytic mixture, the modifier can coordinate to or otherwise interact with transition metal species in the mixture, as well as coordinate to aluminum species. In this respect, in certain embodiments the modifier can modify both the organoaluminum compound and the transition metal complex. Accordingly, while the transition metal complex can be mixed separately with a catalyst modifier prior to adding the transition metal complex to the mixture of organoaluminum compound and the modifier, the presently disclosed subject matter does not require that the transition metal species be mixed with any catalyst modifier prior to adding the transition metal complex to the mixture of organoaluminum compound and the modifier.

As noted above, in certain embodiments, a process of preparing a freeze-dried catalyst composition can include first mixing an organoaluminum compound with a modifier, and then subsequently adding a transition metal complex, to provide a modified catalyst composition. Alternatively, in other embodiments, a process of preparing a freeze-dried catalyst composition can include first mixing an transition metal complex with a modifier, and then subsequently adding an organoaluminum compound, to provide a modified catalyst composition. By way of non-limiting example, a transition metal complex (e.g., titanium tetra-n-butoxide) can be mixed with a modifier (e.g., THF) in a solvent (e.g., hexane). An organoaluminum compound (e.g., triethylaluminum) can then be added to the mixture of transition metal complex and modifier, to provide a modified catalyst composition. The modified catalyst composition can then be further cooled under reduced pressure, to provide a freeze-dried catalyst composition.

As noted above, in certain embodiments, the organoaluminum compound and/or the transition metal complex can optionally be diluted in one or more solvents. Alternatively, the organoaluminum compound and/or transition metal complex can be used without diluting in a solvent before or after they are diluted in or mixed with the modifier. The exemplary process disclosed herein further includes further cooling the modified catalyst composition under reduced pressure, to provide a freeze-dried catalyst composition. The further cooling under reduced pressure can constitute a freeze-drying step. The further cooling can be performed at a temperature of about −200° C. to about 0° C. In certain embodiments, the further cooling can be performed at a temperature of about −80° C. to about −50° C. In other embodiments, the further cooling can be performed at a temperature of about −200° C. to about −80° C. In certain embodiments, the further cooling can be performed at liquid nitrogen temperatures, e.g., at about −196° C. The further cooling can be performed at a reduced pressure of less than ambient pressure, i.e., less than 1 atm. In certain embodiments, the pressure can be less than 0.1 atm, less than 0.01 atm, or less than 0.001 atm. During the further cooling under reduced pressure, relatively volatile substances including excess solvent (e.g., hexane) and modifier (e.g., THF) can be removed via vaporization while the desired catalyst composition remains solid.

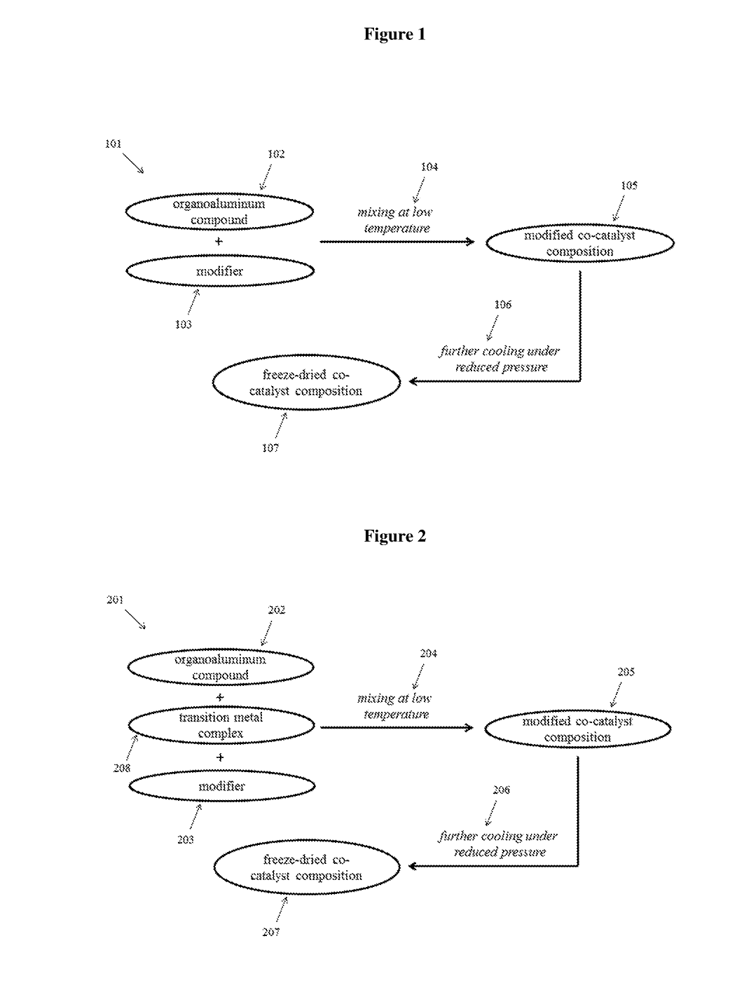

For the purpose of illustration and not limitation, FIG. 2 shows an exemplary process 201 of preparing a freeze-dried catalyst composition. As shown in FIG. 2, the process 201 includes providing an organoaluminum compound 202, a modifier 203, and a transition metal complex 208. The organoaluminum compound 202, the modifier 203, and the transition metal complex 208 can be mixed at low temperature 204 to provide a modified catalyst composition 205. The modified catalyst composition 205 can be further cooled under reduced pressure 206 to provide a freeze-dried co-catalyst composition 207.

The freeze-dried co-catalyst compositions and freeze-dried catalyst compositions disclosed herein can be collected by various techniques known in the art. After collection, the compositions can be maintained under an inert atmosphere for long-term storage. The compositions can optionally be stored at low temperature. The compositions can be stored in solid or waxy form. The compositions can be characterized by various techniques known in the art, e.g., dissolved in an appropriate deuterated solvent and characterized by nuclear magnetic resonance (NMR) spectroscopy.

The freeze-dried catalyst compositions disclosed herein can have improved properties as compared to other catalyst compositions. For example, the freeze-dried catalyst compositions can have well-defined stoichiometry (i.e., molar ratio of organoaluminum compound to modifier and molar ratio of organoaluminum compound to transition metal complex), as any excess modifier can be removed during freeze-drying. Catalyst compositions with well-defined stoichiometry can have improved properties, e.g., improved reproducibility and catalytic activity. Also, the freeze-dried catalyst compositions can be purified as compared to non-freeze-dried catalyst compositions, as solvent and other impurities can be removed during freeze-drying. The freeze-dried catalyst compositions can be waxy or solid in form, which can make them convenient to store and transport. The freeze-dried catalyst compositions can be stored at low temperature and under an inert atmosphere. The freeze-dried catalyst compositions can be stable to storage for extended periods, e.g., for a week, a month, a year, or longer. After storage, the freeze-dried catalyst compositions can be used under standard conditions to catalyze various reactions.

In certain embodiments, the catalyst compositions disclosed herein can be used to prepare homogeneous catalysts rather than heterogeneous catalysts. Accordingly, the organoaluminum compound, the modifier, and the transition metal complex can be mixed and freeze-dried in the absence of any solid support or solid carrier, to provide a composition that does not include a solid support or solid carrier.

The co-catalyst compositions disclosed herein, which include an organoaluminum compound and a modifier, can be used in a wide range of applications. The catalyst compositions, which include an organoaluminum compound, a transition metal complex, and a modifier, can also be used in a wide range of applications. The co-catalyst and catalyst compositions disclosed herein are not limited to applications in olefin oligomerization and polymerization. The co-catalyst and catalyst compositions can be used in a wide range of applications known in the art, including, by way of non-limiting example, diene and alkyne polymerization processes, generation of alkylidene and metal-carbene complexes, transmetallation reactions, carbometallation reactions of alkenes, dienes, and alkynes, and conjugate addition reactions. In certain non-limiting embodiments, the co-catalyst compositions disclosed herein can be used with or without a transition metal complex as Lewis acid catalysts for various reactions in organic chemistry, e.g., epoxide opening, aldol reactions, cross-coupling reactions, conjugate addition reactions, etc. The freeze-dried co-catalyst compositions disclosed herein can be used in many contexts and applications known in the art where an organoaluminum compound is used.

In certain non-limiting embodiments, the compositions disclosed herein can be introduced to a reaction system in at least two or more components, which can be added sequentially. For example, a freeze-dried co-catalyst composition and a transition metal complex diluted in a solvent (e.g., hexane) can be added to the reactor sequentially. In other embodiments, the compositions disclosed herein can be introduced to a reaction system as a single component. For example, a freeze-dried catalyst composition can be added to a reactor.

In a reactor, one or more reactants (e.g., an alkene) can be contacted with a catalyst composition to undergo reaction. In certain embodiments, the freeze-dried co-catalyst composition or the freeze-dried catalyst composition can be dissolved in a solvent prior to addition to the reactor.

By way of non-limiting example, the catalyst compositions disclosed herein can be used for catalytic dimerization of ethylene, e.g., to produce an α-olefin (e.g., 1-butene). Catalytic dimerization of ethylene can be carried out as a continuous reaction or a batch reaction. Catalytic dimerization of ethylene can proceed as a homogeneous reaction (e.g., in the liquid phase), or as a heterogeneous reaction. In some embodiments, catalytic dimerization of ethylene proceeds as a homogeneous liquid phase reaction. In certain non-limiting embodiments, a freeze-dried catalyst composition is dissolved in a solvent (e.g., a hydrocarbon) prior to contacting ethylene with the catalyst composition.

Catalytic dimerization of ethylene can be performed at a temperature of from about 20° C. to about 150° C., from about 40° C. to about 100° C., from about 20° C. to about 70° C., from about 50° C. to about 70° C., from about 50° C. to about 55° C., or from about 55° C. to about 65° C. In some embodiments, catalytic dimerization of ethylene is performed at a temperature of about 60° C. Catalytic dimerization of ethylene can be performed at a pressure of from about 5 bars to about 50 bars, from about 10 bars to about 40 bars, or from about 15 bars to about 30 bars. Catalytic dimerization of ethylene can be conducted in a batch, a selected volume of the presently disclosed catalyst composition can be introduced into a reactor provided with usual stirring and cooling systems, and can be subjected therein to an ethylene pressure, which can be from about 22 bars to about 27 bars. In some embodiments, catalytic dimerization of ethylene using the presently disclosed catalyst composition is conducted at an ethylene pressure of about 23 bar. One of ordinary skill in the art can adjust the temperature, pressure and other conditions of the reaction in order to bring about favorable properties of the reaction, for example, in order to ensure that the reaction system is present as a homogeneous liquid phase. The reaction product (e.g., 1-butene) can be extracted by any methods which one of ordinary skill in the art would consider to suitable in the context of the presently disclosed subject matter. Suitable methods of extraction include, but are not limited to, distillation, precipitation, crystallization, and membrane permeation.

Catalytic dimerization of ethylene performed using the presently disclosed catalyst compositions can have advantages over existing methods. For example, in certain embodiments, ethylene can be dimerized to 1-butene with a shortened induction period, extended lifetime of the catalyst composition, improved catalyst stability, improved selectivity for the desired product, reduced formation of polymer side products, reduced fouling, and overall improved catalyst activity. For example, certain existing commercial processes of dimerization of ethylene to produce 1-butene, e.g., the Alphabutol™ system, are characterized by long induction periods during which negligible amounts of ethylene are consumed. When existing commercial processes of dimerization of ethylene to produce 1-butene, e.g., the Alphabutol™ system, are used on plant scale, the induction periods can be many hours long. In certain embodiments, catalyst compositions including organoaluminum co-catalysts modified by a modifier can be characterized by shorter induction periods on lab scale and on plant scale, e.g., induction periods of less than 5 hours, less than 3 hours, less than 2 hours, less than 1 hour, less than 30 minutes, less than 15 minutes, less than 10 minutes, less than 5 minutes, less than 3 minutes, less than 2 minutes, or less than 1 minute.

The processes disclosed herein for modifying organoaluminum co-catalysts, preparing freeze-dried co-catalyst and catalyst compositions, and performing various catalytic processes can be coupled to various further subsequent reactions in order to obtain downstream products. By way of non-limiting example, the process disclosed herein for catalytic dimerization of ethylene to produce 1-butene can be coupled to further subsequent reactions in order to obtain downstream products. Downstream products are those obtained from oligomerization reactions, polymerization reactions, hydrogenation reactions, halogenation reactions, and other chemical functionalization reactions. The chemical functionalization products can be aromatic or non-aromatic compounds, saturated or unsaturated compounds, ketones, aldehydes, esters, amides, amines, carboxylic acids, alcohols, etc. Monomeric downstream products can be chloro-butene, butadiene, butanol, and butanone. In certain embodiments, the downstream products are those obtained from polymerization reactions. Polymerization reactions can be mono-polymerization reactions or co-polymerization reactions. The polymerization product can be poly-butene. Co-polymers can include α-olefin (e.g., 1-butene) and one or more co-monomers including, but not limited to: ethylene, propene, pentene, styrene, acrylic acid, vinyl chloride. In certain embodiments, the co-polymer is a co-polymer of ethylene and 1-butene. The ethylene monomers can be present in a larger wt. % the than the 1-butene monomers in the co-polymer. For example, the weight ratio of ethylene monomers to 1-butene monomers can be from about 50:1 to about 5:1, from about 30:1 to about 10:1, or from about 25:1 to about 15:1. One or ordinary skill in the art can vary the ratio relating the mass of ethylene monomers and 1-butene monomers in order to tune the desired properties of polyethylene or polypropylene, such as crystallinity and elasticity.

In some embodiments of the process of preparing a downstream product, the product includes compounds with chain lengths in proportions determined by or approximating to the Anderson Schulz Flory distribution (see P. L. Spath and D. C. Dayton. “Preliminary Screening—Technical and Economic Assessment of Synthesis Gas to Fuels and Chemicals with Emphasis on the Potential for Biomass-Derived Syngas”, NREL/TP510-34929, December, 2003, pp. 95).

In some embodiments, the downstream products are further connected to yield fatty acids, e.g., with chain lengths in proportions determined by or approximating to the Anderson Schulz Flory distribution.

In some embodiments, the downstream products are further processed, particularly in the case where the downstream product is a polymer, particularly when it is a polyethylene derivative. In one aspect of this embodiment, this further processing can involve formation of shaped objects such as plastic parts for electronic devices, automobile parts, such as bumpers, dashboards, or other body parts, furniture, or other parts or merchandise, or for packaging, such as plastic bags, film, or containers.

The term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

All publications, patents and patent applications cited herein are hereby expressly incorporated by reference for all purposes to the same extent as if each was so individually denoted.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

1. A process of preparing a freeze-dried co-catalyst composition, the process comprising:
mixing an organoaluminum compound with a modifier at low temperature, to provide a modified co-catalyst composition; and
further cooling the modified co-catalyst composition under reduced pressure, to provide a freeze-dried co-catalyst composition.

2. The method of claim 1, further comprising
mixing a transition metal complex with the organoaluminum compound and the a modifier at low temperature, to provide a modified catalyst composition; and
further cooling the modified catalyst composition under reduced pressure, to provide a freeze-dried catalyst composition.

3. The method of claim 1, further comprising
mixing the freeze-dried co-catalyst composition with a transition metal complex, to provide a catalyst composition.

4. A freeze-dried co-catalyst composition made by the process of claim 1.

5. A freeze-dried catalyst composition made by the process of claim 2.

6. A catalyst composition made by the process of claim 3.

7. A freeze-dried co-catalyst composition comprising an organoaluminum compound and a modifier.

8. The composition of claim 7, wherein the modifier is a compound that decreases the initial reducing strength of the organoaluminum compound.

9. The composition of claim 8, wherein the modifier is an ether.

10. The composition of claim 9, wherein the ether is tetrahydrofuran.

11. The composition of claim 7, wherein the composition does not comprise a solid support or solid carrier.

12. A freeze-dried catalyst composition comprising an organoaluminum compound, a transition metal complex, and a modifier.

13. The composition of claim 12, wherein the transition metal complex is titanium tetra-n-butoxide.

14. The composition of claim 12, wherein the composition does not comprise a solid support or solid carrier.

15. A process of preparing an α-olefin, comprising:
providing a freeze-dried catalyst composition comprising an organoaluminum compound, a transition metal complex, and a modifier; and
contacting an alkene with the freeze-dried catalyst composition to obtain an α-olefin.

16. The process of claim 15, further comprising dissolving the freeze-dried catalyst composition in a solvent prior to contacting the alkene with the freeze-dried catalyst composition.

17. The process of claim 15, wherein the modifier is an ether.

18. The process of claim 15, wherein the ether is tetrahydrofuran.

19. The process of claim 15, wherein the transition metal complex is titanium tetra-n-butoxide.

20. The process of claim 15, wherein the composition does not comprise a solid support or solid carrier.
